SEQUENCE LISTING (1 sequence)

SEQ ID NO: 1 - protein, 23 aa, homo sapiens
MDEEEDGAGAEESGQPRSFMRLC

(19) United States
(12) Patent Application Publication
(10) Pub. No.: US 2006/0003020 A1
(43) Pub. Date: Jan. 5, 2006

(54) Anti-metastatic ability of mibefradil and gadolinium

(75) Inventors: Howard R. Petty, Livonia, MI (US); Andrei L. Kindzelski, Grosse Point, MI (US); Ji-Biao Huang, Ann Arbor, MI (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: THE REGENTS OF THE UNIVERSITY OF MICHIGAN, Ann Arbor, MI (US)

(21) Appl. No.: 11/077,088

(22) Filed: Mar. 10, 2005

Related U.S. Application Data
(60) Provisional application No. 60/552,086, filed on Mar. 11, 2004.

(51) Int. Cl. A61K 31/4965; A61K 31/455; A61K 31/445; A61K 31/4164

(52) U.S. Cl. 424/646; 514/355; 514/389; 514/255.06; 514/317

(57) ABSTRACT

The present invention relates to methods for inhibiting tumor cell metastasis in cancer comprising administering a composition comprising a T-type calcium channel inhibitor, such as mibefradil.

The present application claims the priority benefit of U.S. Provisional Application No. 60/552,086, filed Mar. 11, 2004, incorporated herein by reference in its entirety.

### FIELD OF THE INVENTION

The present invention relates to the treatment of cancer metastasis using mibefradil, an inhibitor of T-type calcium channels involved in cell motility. The invention further provides for the administration of T-type calcium channel inhibitors in conjunction with non-voltage gated (NVG) calcium channel inhibitors, as well as in combination with other chemotherapeutic or radiotherapeutic treatment regimens, to inhibit tumor cell metastasis.

### BACKGROUND OF THE INVENTION

The invasion and metastasis of tumor cells require cell migration (Mareel et al., Physiol Rev. 83: 337-376, 2003; Entschladen et al., J. Cancer Res. Clin Oncol. 126: 671-681, 2000). Cell migration is a cyclic process involving the repetitive extension of invadopodia/lamellipodia at the leading edge of the cell, the formation of adhesion sites, contraction of the cell body, and the release of trailing adhesion sites. The cyclic morphological and adherence changes observed during cell migration are accompanied by repetitive Ca²⁺ signals, which take the form of Ca²⁺ spikes or oscillations. Ca²⁺ transients have been observed in migrating neurons, neutrophils, fibroblasts, eosinophils, tumor cells and other cell types (Komuro et al., Neuron 17 275-85, 1996; Mandeville et al., Biophys. J. 68: 1207-17, 1995; Hahn et al., Nature 359: 736-38, 1992; Brundage et al., Science, 254: 703-706, 1991; Giannone et al., J. Biol. Chem., 277: 26364-71, 2002; Ronde et al., Biochim Biophys Acta, 1498: 273-280, 2000). These oscillations are thought to participate in coordinating the cyclic temporal features of cell migration, such as pseudopod extension, actin assembly, integrin regulation; the phosphorylation-mediated regulation of focal adhesion formation, and pericellular proteolysis (Mareel et al., supra; Entschladen supra; Komuro et al supra; Mandeville et al., supra; Hahn et al., supra; Brundage et al., supra; Giannone et al., supra; Ronde et al., supra). Thus, repetitive Ca²⁺ signals likely play an important role in the repetitive structural and functional changes required for cell movement.

Intracellular Ca²⁺ spikes may involve Ca²⁺ release from intracellular stores as well as Ca²⁺ entry from the extracellular environment. Ca²⁺ entry from the extracellular environment is mediated by Ca²⁺-permeable ion channels of the plasma membrane, of which the voltage-gated Ca²⁺ channels are best characterized (Hofmann et al., Rev. Physiol. Biochem. Pharmacol. 139:33-87, 1999). Five types of high voltage-activated (HVA) Ca²⁺ channels (named L-, N-, P-, Q-, and R-type) and one type of low voltage-activated (LVA) Ca²⁺ channel (known as T-type) have been identified based on the pharmacological and biophysical characteristics of their currents. HVA channels are broadly distributed on neurons (N'Gouemo et al., Neuroscience, 120:815-826, 2003), and myocytes (Benitah et al., Basic Res. Cardiol., 97 Suppl 1:111-8, 2002) where they participate in functions such as neuronal excitability and muscle contraction.

T-type Ca²⁺ channels are found on many cell types, including fibroblasts, neurons, smooth muscle cells, and thyroid cells (Perez-Reyes et al., Physiol. Rev., 83: 117-161, 2003) where they serve to limit the electrical load and transiently activate cells as a “pacemaker” (Perez-Reyes et al., Physiol. Rev., 83:117-161, 2003; McDonald et al., Physiol. Rev., 74: 365-507, 1994). T-type calcium channels are also expressed on cell lines isolated from tumor cells (Perez-Reyes et al., supra). Results from the study of T-type currents on fibroblasts and other cell lines, such as NG108-15 neuronal cells, demonstrate that these cells have different T-type calcium channel kinetics, leading to the suggestion that multiple T-type channel isoforms exist. Differences in the pharmacology of T-type channels also suggested the existence of distinct channel isoforms (Herrington et al., J Neurophysiol 68:213-232, 1992). Multiple low-voltage-activated calcium channel Caᵥ3 α1 subunits (α1G, α1H and α1I) have been cloned, which differ in their kinetics, pharmacology, and recovery from inactivation (Klockner et al., Eur. J. Neurosci. 11:4171-4178, 1999; Lee et al., Biophys. J. 77:3034-3042, 1999).

Non-voltage-gated (NVG) Ca²⁺ influx channels are activated upon binding intracellular and/or extracellular messengers, chemical or mechanical stress, and by the Ca²⁺ level of intracellular stores (Hi1le, B. Ionic channels of excitable membranes. Sunderland; MA, Sinauer, 2001). Among these channels, transient receptor potential (TRP) and TRP-like (TRPL) channels are molecularly identified and distributed in a variety of tissues and species (Vennekens et al., Cell Calcium 31.:53-64, 2002). TRPs are likely identical to several NVG channels such as: store-operated channels (SOC) (Montell et al., Mol. Pharmacol., 52: 755-63, 1997; Clapham et al., Nat. Rev. Neurosci. 2:387-396, 2001), mechanosensitive cation channels (Welsh et al., Circ. Res. 90:248-250, 2002), and receptor-activated cation channels (Jung et al., Am. J. Physiol. 282.C347-0359, 2002). The specific physiological functions of TRP and TRPL channels have not been rigorously established.

Cell adhesion and migration require transient cell activation and rapid inactivation, which is usually the function of T-type Ca²⁺ channels and TRP/TRPL channels (Hardie et al., Proc. R. Soc. Lond. B Biol. Sci. 245:203-10, 1991; Scott et al., Cell 91:375-83, 1997). Consistent with this, studies have suggested that the LVA (T-type) calcium channel blocker, mibefradil, effectively inhibits leukocyte adhesion and locomotion (Blaheta et al., Immunology 94.213-220, 1998; Nebe et al., Cardiovasc. Drugs Ther. 16:183-193, 2002).

Mibefradil, a non-dihydropyridine compound, was found to inhibit T-type calcium channels 10 to 30 times more selectively than L-type, high voltage gated channels (Perez-Reyes, supra). This unique interaction led to the development of mibefradil as a therapeutic calcium channel blocker useful as an antihypertensive to relieve high blood pressure, and as a treatment for heart conditions such as angina pectoris (Krayenbuhl et al., Eur J Clin Pharmacol 55: 559-565, 1999). Other T-type channel inhibitors which are less effective than mibefradil in treating heart disease include the dihydropyridines amlodipine, felodipine, and flunarizine. Mibefradil analogs have been developed which demonstrate similar T-type channel inhibitory function, but exhibit fewer negative drug interactions. Such analogs include R040-6040 (Eller et al., Br. J. Pharmacol. 130:669-77, 2000) and lipophilic mibefradil analogs.

T-type channel blockers have also been developed for the treatment of various neurological disorders such as epilepsy and psychotic disorders (Perez-Reyes, supra; Coulter et al., Ann Neurol 25: 582-593, 1989; Enyeart et al., Mol Pharmacol 42:364-372, 1992). T-type calcium channel regulation may also occur through hormones or hormonal regulators and via neurotransmitters in isolated neurons (Osipenko et al., Endocrinology 134:511-514, 1994; Chen et al., J Physiol. 425: 29-42, 1990; Schroeder et al., Neuron 6: 13-20, 1991).

In vitro and in vivo studies have suggested that Ca²⁺ channels are important in tumor cell proliferation and invasion (e.g., Wu et al., Clin. Cancer Res. 3: 1915-1921, 1997; Alessandro et al., In Vivo 10:153-160, 1996; Vilpo et al., Haematologica 85:806-813, 2000; Gaffen et al., J. Pharm. Pharmacol. 43:401-405, 1991; Kohn et al., Clin. Cancer Res. 7:1600-1609, 2001), and also suggested that Ca²⁺ waves are present in tumor cells (Ronde et al., supra). Carboxyamidotriazole (CAI), an inhibitor of NVG Ca²⁺ channels, induces tumor cell apoptosis (Ge et al., Clin. Cancer Res. 6. 1248-1254, 2000) and inhibits tumor cell growth and invasion (Wu et al., Clin. Cancer Res. 3: 1915-1921, 1997; Bauer et al., J. Pharmacol. Exp. Ther. 292:31-37, 2000), implying that NVG Ca²⁺ channels are closely related with tumor cell physiology. Additionally, verapamil, an HVA Ca²⁺ channel blocker, has been used in vitro to treat cancer cells, and in combined therapy approaches (e.g., Rybalchenko et al., Mol. Pharmacol. 59:1376-1387, 2001; Farias et al., Int. J. Cancer 78:727-734, 1998.). Verapamil given at higher doses, however, also blocks potassium channels (Robe et al., Br. J. Pharmacol. 13:1275-1284, 2000), which leads to ambiguity in its mechanism of action.

Studies demonstrate that T-type calcium channels expressed in fibroblast cells exhibit unique regulation compared to other cells expressing T-type calcium channels (Pemberton et al., Pflügers Arch. 440: 452-461, 2000). Aberrant regulation of fibroblasts in areas of soft tissue or bone is a primary cause of firbosarcoma type tumors.

Surgery and removal of the cancerous tissue is the most common form of treatment for fibrosarcoma. However, fibrosarcomas of particular origin show a high rate of recurrence and metastasis after surgery (Papagelopoulos et al., Am. J. Orthop. 31:253-7, 2002; Yamaguchi et al., Clin Oral Investig. 2003 Oct. 30; Antonescu et al., Am. J. Surg. Pathol. 25:699-709, 2001) which complicates complete removal of the cancer in a patient.

Given the strong potential for fibrosarcoma to recur and/or metastasize in an individual diagnosed with this type of cancer, and the diverse nature of each type of fibrosarcoma, there is a need in the art to develop more effective methods for inhibiting or ameliorating recurring and metastasizing fibrosarcoma.

Thus, there remains a need in the art to identify methods for inhibiting tumor cell signaling and tumor cell metastasis based on calcium flux, which regulates tumor cell migration and invasion. Further, there remains a need to identify calcium channel inhibitors which can ameliorate tumor cell metastasis and potentiate the effects of other chemotherapeutic and radiotherapeutic cancer treatment regimens.

### SUMMARY OF THE INVENTION

The present invention addresses the need in the art to provide methods for effectively inhibiting tumor cell metastasis in a subject. Accordingly, the invention provides methods for inhibiting tumor cell metastasis using T-type calcium channel inhibitor compositions. The invention further provides methods for designing a regimen for treating and/or preventing tumor cell metastasis using compositions comprising a T-type calcium channel inhibitor.

In one aspect, the invention contemplates methods of inhibiting tumor cell metastasis in cancer comprising the step of administering a T-type calcium channel inhibitor in an amount effective to inhibit tumor cell metastasis. In one aspect, the T-type calcium channel comprises a Caᵥ3 α1 subunit, wherein the al subunit is selected from the group consisting of α₁G, α₁H and α₁I.

In one embodiment, the T-type calcium channel inhibitor is selected from the group consisting of a diphenyubutylpiperidine, a hormone or hormonal regulator, an antiepileptic agent, an antipsychotic agent, and efonidipine. It is contemplated that the diphenyubutylpiperidine is selected from the group consisting of penfluridol and fluspiriline. The invention also contemplates inhibition of T-type calcium channels with hormones or hormonal regulators selected from the group consisting of a GABA agonist, e.g., baclofen, or neurotransmitters, such as dopamine, serotonin, somatostatin, and opiods. It is further contemplated that an antiepileptic agent useful as a T-type calcium channel inhibitor is selected from the group consisting of ethosuximide, methyl-phenylsuccinimide, phenyloin, and zonisamide. In a related embodiment, a T-type calcium channel is inhibited by an anti-psychotic agents selected from the group consisting of penfluridol, fluspiriline, thioridazine, clozapine, and haloperidol.

In another embodiment, the T-type calcium channel inhibitor is selected from the group consisting of mibefradil, nickel (Ni²⁺), baclofen, flunarizine, nicardipine, felodipine, amiloride ethosuximide, methyl-phenylsccinimide, phenyloin, zonisamide, and penfluridol. In a preferred embodiment, the T-type calcium channel inhibitor is mibefradil.

The method of the invention further contemplates a method of inhibiting tumor cell metastasis in cancer wherein the T-type calcium channel inhibitor is administered in combination with one or more additional therapeutics/second agent, wherein the second agent is a second calcium channel blocker, a cytokine or growth factor, a chemotherapeutic agent, a radiotherapeutic agent, or radiation therapy. In one aspect, it is contemplated that the second agent is administered simultaneously or concurrently with the T-type calcium channel inhibitor. It is further contemplated that the second agent is administered either prior to or subsequent to administration of the T-type calcium channel inhibitor.

In one aspect of the invention, the T-type calcium channel inhibitor is administered in combination with a cytokine or growth factor. The cytokine or growth factor may be any factor that is useful in inhibiting tumor metastasis and may be selected from the cytokines or growth factors described herein.

In a related aspect, the T-type calcium channel inhibitor is administered in conjunction with a chemotherapeutic or radiotherapeutic agent. The chemotherapeutic agent or radiotherapeutic agent may be a member of the class of agents including an anti-metabolite; a DNA-damaging agent; a cytokine or growth factor; a covalent DNA-binding drug; a topoisomerase inhibitor; an anti-mitotic agent; an anti-tumor antibiotic; a differentiation agent; an alkylating agent; a methylating agent; a hormone or hormone antagonist; a nitrogen mustard; a radiosensitizer; and a photosensitizer. Specific examples of these agents are described elsewhere in the application.

The invention further provides a method wherein the T-type calcium channel inhibitor is administered in combination with a NVG calcium channel inhibitor. In one embodiment, the NVG calcium channel is selected from the group from transient receptor potential (TRP) or TRP-like (TRPL) channel, a store-operated channels (SOC), a mechanosensitive cation channel, and a receptor-activated cation channel. In another embodiment, the NVG calcium channel inhibitor is a transient receptor potential (TRP) or TRP-like (TRPL) channel.

In a related embodiment, the NVG calcium channel inhibitor is selected from the group consisting of carboxyamidotriazole (CAI), gadolinium (Gd³⁺), SKF-96365, econazole, YM58483 and La³⁺.

In a further embodiment, the T-type calcium channel inhibitor administered is mibefradil and the NVG calcium channel inhibitor is gadolinium (Gd³⁺). In another embodiment, the T-type calcium channel inhibitor is mibefradil and the NVG calcium channel inhibitor is carboxyamido-triazole (CAI). It is contemplated that the T-type calcium channel inhibitor and the NVG calcium channel inhibitor are administered in combination with a cytokine or growth factor. In a further embodiment, the T-type calcium channel inhibitor and the NVG calcium channel inhibitor are administered in combination with a chemotherapeutic or radiotherapeutic agent.

It is contemplated that the T-type calcium channel inhibitor and the NVG calcium channel inhibitor are administered simultaneously, in the same formulation. It is further contemplated that the T-type calcium channel inhibitor and the NVG calcium channel inhibitor are administered at different times. In one embodiment, the T-type calcium channel inhibitor and the NVG calcium channel inhibitor are administered concurrently. In a second embodiment, the T-type calcium channel inhibitor is administered prior to the NVG calcium channel inhibitor. In a third embodiment, the T-type calcium channel inhibitor is administered subsequent to the NVG calcium channel inhibitor.

The invention contemplates a pharmaceutical composition comprising a T-type calcium channel inhibitor and a pharmaceutically acceptable carrier, diluent or excipient. In one embodiment, the T-type calcium channel inhibitor composition comprises mibefradil in combination with a second agent such as a second calcium channel inhibitor, wherein the second calcium channel inhibitor is either a T-type or a NVG calcium channel inhibitor. In related embodiments, the composition comprises mibefradil in combination with a second agent such as a chemotherapeutic agent; or mibefradil in a pharmaceutical composition comprising a growth factor or cytokine. Mibefradil analogs are also contemplated for use in a mibefradil composition.

Generally, compositions of the invention are those that will inhibit tumor cell metastasis at lower concentrations, thereby permitting use of the modulators in a pharmaceutical composition at lower effective doses. Such compositions are suitable for administration by several routes such as intrathecal, parenteral, topical, intranasal, inhalational, oral administration, or any other clinically acceptable route of administration.

The invention provides methods for treating tumor cell metastasis in a subject having cancer. In one aspect, the cancer being treated is any form of cancer as described herein, including carcinomas, sarcomas, leukemias, and lymphomas. Specific examples of these cancers contemplated by the methods of the invention are set out below. In one embodiment, the cancer is fibrosarcoma.

The subject treated by the methods of the invention may be human, or any non-human animal model for human medical research, or an animal of importance as livestock or pets, (e.g., companion animals). In one variation, the subject has a disease or condition characterized by a need for amelioration or elimination of tumor cell metastasis, and administration of a composition comprising a T-type channel inhibitor improves the animal's state, for example, by palliating disease symptoms, slowing tumor cell metastasis, eliminating tumor cell metastasis, or otherwise improving clinical symptoms. In a preferred embodiment, the subject to be treated is human.

The present invention also provides methods for designing a treatment regimen to inhibit or reduce metastasis in a patient. It is contemplated that the therapeutic regimen is developed using cancer cells isolated from an individual and tested in the presence and absence of a mibefradil composition to determine a mibefradil composition most effective at inhibiting tumor metastasis in said individual. It is contemplated that the invention provides a method for designing a treatment regimen for a patient with tumor cell metastasis comprising the steps of: (a) isolating a cell from said patient, wherein said cell comprises a T-type calcium channel; (b) contacting said cell with a mibefradil composition; (c) detecting T-type calcium channel activity in said cell to determine the amount of T-type calcium channel activity in the presence and in the absence of the mibefradil composition; and (d) designing a treatment regimen for said patient which includes administration of a mibefradil composition that specifically inhibits T-type calcium channel activity in said patient.

### DETAILED DESCRIPTION OF THE INVENTION

The present invention is based, in part, on the discovery of activity of mibefradil on tumor cell growth. Methods of the invention for the treatment of tumor cell metastasis include mibefradil's use alone or in combination with other therapeutic agents, the biological effects of which, alone or in combination, were not fully appreciated. The present invention discloses a role for T-type Ca²⁺ channel blockers in inhibiting tumor metastasis, and a role for NVG calcium channels in calcium signaling in tumor cells, a role which has not been previously reported. These observations show that mibefradil enhances the anti-metastatic activity of chemotherapeutic agents, and provides a means to improve new or known anti-metastatic drugs which, when used in combination with mibefradil, inhibit tumor cell metastasis.

In order that the invention may be more completely understood, several definitions are set forth.

As used herein, “potentiate” or “synergize” refers to activity of mibefradil, which, when given in conjunction with either a second calcium channel inhibitor or chemotherapeutic agent, enhances inhibition of tumor metastasis beyond that of administration of mibefradil alone.

A “therapeutically effective amount” or “effective amount” refers to that amount of the compound sufficient to result in amelioration of symptoms, for example, treatment, healing, prevention or amelioration of the relevant medical condition, or an increase in rate of treatment, healing, prevention or amelioration of such conditions. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

“Mibefradil composition” as used herein refers to compositions that comprise mibefradil. Mibefradil compositions contemplated for use in the invention include mibefradil alone in a pharmaceutically acceptable carrier, mibefradil in combination with a second agent such as a second calcium channel inhibitor, or mibefradil in combination with a second agent such as a chemotherapeutic or radiotherapeutic agent, which can be, but is not limited to, any agent set out in Table 1. Mibefradil composition also includes mibefradil in a pharmaceutical composition comprising a growth factor or cytokine as set out below. Mibefradil analogs are also contemplated for use in a mibefradil composition.

It will be understood by those of skill in the art that the terms “calcium channel inhibitor” and “calcium channel blocker” can be used interchangeably.

Treatment of Cancer Using the Methods of the Invention

The present invention provides methods of treating cancer in an animal, comprising administering to the animal an effective amount of a composition that inhibits tumor cell activity. The invention is further directed to methods of inhibiting cancer cell growth, including processes of cellular proliferation, invasiveness, and metastasis in biological systems. Methods include administration of a mibefradil composition as an inhibitor of cancer cell growth. The methods, therefore, are employed to inhibit or reduce cancer cell growth, invasiveness, metastasis, or tumor incidence in living animals, such as mammals.

The cancers treatable by methods of the present invention preferably occur in mammals. Mammals include, for example, humans and other primates, as well as pet or companion animals such as dogs and cats, laboratory animals such as rats, mice and rabbits, and farm animals such as horses, pigs, sheep, and cattle.

Tumors or neoplasms include growths of cells in which the multiplication of the cells is uncontrolled and progressive. Some such growths are benign, but others are malignant and may lead to death of the organism. Malignant neoplasms or cancers are distinguished from benign growths in that, in addition to exhibiting aggressive cellular proliferation, they may invade surrounding tissues and metastasize. Moreover, malignant neoplasms are characterized in that they show a greater loss of differentiation (greater “dedifferentiation”), and of their organization relative to one another and their surrounding tissues. This property is also called “anaplasia.”

Neoplasms treatable by the present invention include solid tumors, for example, carcinomas and sarcomas. Carcinomas include malignant neoplasms derived from epithelial cells which infiltrate, for example, invade, surrounding tissues and give rise to metastases. Adenocarcinomas are carcinomas derived from glandular tissue, or from tissues that form recognizable glandular structures. Another broad category of cancers includes sarcomas and fibrosarcomas, which are tumors whose cells are embedded in a fibrillar or homogeneous substance, such as embryonic connective tissue. The invention also provides methods of treatment of cancers of myeloid or lymphoid systems, including leukemias, lymphomas, and other cancers that typically are not present as a tumor mass, but are distributed in the vascular or lymphoreticular systems.

Fibrosarcomas are defined as a malignant tumors of soft tissue (e.g. fat, muscle tendons, nerves, joint tissue, and blood vessels) or bone which comprise tissue from collagen-producing fibroblasts. These tumors often have the capacity to spread beyond their original location and invade other surrounding soft tissue sites. Numerous types of fibrosarcomas have been identified to date, and are categorized as either adult fibrosarcoma or congenital and infantile fibrosarcoma. Malignant fibrous histiocytoma, fibromatosis and nodular fascitis have recently been recognized as specific types of fibrosarcoma. Additional fibrosarcomas include low grade fibromyxoid sarcoma, hyalinizing spindle cell tumor with giant rosettes, sclerosing epithelioid fibrosarcoma and congenital and infantile fibrosarcoma. All of these tumor types are characterized by tumors comprising spindle-shaped, transformed fibroblast cells. Some fibrosarcomas are also classified as inflammatory myofibroblastic tumors, such as low grade myofibroblastic sarcoma and high grade myofibroblastic sarcoma. These cancers are also characterized by tumors comprising spindle-shaped cells.

Further contemplated are methods for treatment of adult and pediatric oncology, growth of solid tumors/malignancies, myxoid and round cell carcinoma, locally advanced tumors, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma lung cancer (including small cell carcinoma of the lungs, cutaneous T cell lymphoma, Hodgkin's lymphoma, non-Hodgkin's lymphoma, cancer of the adrenal cortex, ACTH-producing tumors, non-small cell lung cancers, breast cancer, including small cell carcinoma and ductal carcinoma), gastro-intestinal cancers (including stomach cancer, colon cancer, colorectal cancer, and polyps associated with colorectal neoplasia), pancreatic cancer, liver cancer, urological cancers (including bladder cancer, such as primary superficial bladder tumors, invasive transitional cell carcinoma of the bladder, and muscle-invasive bladder cancer), prostate cancer, malignancies of the female genital tract (including ovarian carcinoma, primary peritoneal epithelial neoplasms, cervical carcinoma, uterine endometrial cancers, vaginal cancer, cancer of the vulva, uterine cancer and solid tumors in the ovarian follicle), malignancies of the male genital tract (including testicular cancer and penile cancer), kidney cancer (including renal cell carcinoma, brain cancer (including intrinsic brain tumors, neuroblastoma, astrocytic brain tumors, gliomas, and metastatic tumor cell invasion in the central nervous system), bone cancers (including osteomas and osteosarcomas), skin cancers (including malignant melanoma, tumor progression of human skin keratinocytes, basal cell carcinoma, and squamous cell cancer), thyroid cancer, retinoblastoma, neuroblastoma, peritoneal effusion, malignant pleural effusion, mesothelioma, Wilms's tumors, gall bladder cancer, trophoblastic neo-plasms, hemangiopericytoma, and Kaposi's sarcoma.

A combination of a T-type calcium channel inhibitor, such as mibefradil, with one or more additional therapeutics/second agents in methods of the invention may reduce the amount of either agent needed to be a therapeutically effective dosage, and thereby reduce any negative side effects the agents may induce in vivo. Additional therapeutics or second agents contemplated for use in combination with a T-type calcium channel inhibitor, such as mibefradil, include a second calcium channel inhibitor, a growth factor or cytokine, a chemotherapeutic agent, a radiotherapeutic agent, or radiation therapy.

Any chemotherapeutic or radiotherapeutic agent may be suitable for use in combination with mibefradil in a method of the invention, and may be identified by means well known in the art. Examples of suitable chemotherapeutic and radiotherapeutic agents include, but are not limited to: an anti-metabolite; a DNA-damaging agent; a cytokine or growth factor useful as a chemotherapeutic agent; a covalent DNA-binding drug; a topoisomerase inhibitor; an anti-mitotic agent; an anti-tumor antibiotic; a differentiation agent; an alkylating agent; a methylating agent; a hormone or hormone antagonist; a nitrogen mustard; a radiosensitizer; a photosensitizer; a radiation source, optionally together with a radiosensitizer or photosensitizer; or other commonly used therapeutic agents.

Specific examples of chemotherapeutic agents useful in methods of the present invention are listed in Table 1.

Mibefradil may also be administered in conjunction with a second T-type calcium channel inhibitor or high voltage activated or non-voltage gated calcium channel inhibitor. Other T-type calcium channel inhibitors which are less effective than mibefradil in treating heart disease, but are contemplated for use herein, include the dihydropyridines amlodipine, felodipine, and flunarizine. Mibefradil analogs have been developed which demonstrate similar T-type channel inhibitory function, but exhibit fewer negative drug interactions. Such analogs include R040-6040 (Eller et al., Br. J. Pharmacol. 130:669-77, 2000.) and lipophilic mibefradil analogs.

T-type channel blockers developed for the treatment of neurological disorders such as epilepsy and psychotic disorders are also useful in the methods of the invention. In the invention, antiepileptic agents useful as T-type calcium channel inhibitors include ethosuximide, methyl-phenylsuccinimide, phenyloin, and zonisamide. Antipsychotic agents useful as T-type calcium channel inhibitors include diphenyubutylpiperidines compounds, such as penfluridol and fluspiriline. Additional antipsychotic T-type calcium channel inhibitors in use clinically, but less effective than penfluridol, include thioridazine, clozapine, and haloperidol.

T-type calcium channels are also regulated through hormones or hormonal regulators. For example, the GABA agonist baclofen, or neurotransmitters, such as dopamine, serotonin, somatostatin, and opiods.

TABLE 1

Alkylating agents
Nitrogen mustards
mechlorethamine
cyclophosphamide
ifosfamide
melphalan
chlorambucil
Nitrosoureas
carmustine (BCNU)
lomustine (CCNU)
semustine (methyl-CCNU)
Ethylenimine/Methyl-melamine
thriethylenemelamine (TEM)
triethylene thiophosphoramide
(thiotepa)
hexamethylmelamine
(HMM, altretamine)
Alkyl sulfonates
busulfan
Triazines
dacarbazine (DTIC)
Antimetabolites
Folic Acid analogs
methotrexate
Trimetrexate
Pemetrexed
Multi-targeted antifolate
Pyrimidine analogs
5-fluorouracil
fluorodeoxyuridine
gemcitabine
cytosine arabinoside
(AraC, cytarabine)
5-azacytidine
2,2′-difluorodeoxy-cytidine
Purine analogs
6-mercaptopurine
6-thioguanine
azathioprine
2′-deoxycoformycin
(pentostatin)
erythrohydroxynonyl-adenine
(EHNA)
fludarabine phosphate
2-chlorodeoxyadenosine
(cladribine, 2-CdA)
Type I Topoisomerase
Inhibitors
camptothecin
topotecan
irinotecan
Natural products
Antimitotic drugs
paclitaxel
Vinca alkaloids
vinblastine (VLB)
vincristine
vinorelbine
Taxotere ® (docetaxel)
estramustine
estramustine phosphate
Epipodophylotoxins
etoposide
teniposide
Antibiotics
actimomycin D
daunomycin (rubido-mycin)
doxorubicin (adria-mycin)
mitoxantroneidarubicin
bleomycinsplicamycin
(mithramycin)
mitomycinC
dactinomycin
Enzymes
L-asparaginase
Biological response modifiers
interferon-alpha
IL-2
G-CSF
GM-CSF
Differentiation Agents
retinoic acid derivatives
Radiosensitizers
metronidazole
misonidazole
desmethylmisonidazole
pimonidazole
etanidazole
nimorazole
RSU 1069
EO9
RB 6145
SR4233
nicotinamide
5-bromodeozyuridine
5-iododeoxyuridine
bromodeoxycytidine
Miscellaneous agents
Platinium coordination complexes
cisplatin
Carboplatin
oxaliplatin
Anthracenedione
mitoxantrone
Substituted urea
hydroxyurea
Methylhydrazine derivatives
N-methylhydrazine (MIH)
procarbazine
Adrenocortical suppressant
mitotane (o, p′- DDD)
ainoglutethimide
Cytokines
interferon (*, *, *)
interleukin-2
Hormones and antagonists
Adrenocorticosteroids/antagonists
prednisone and equivalents
dexamethasone
ainoglutethimide
Progestins
hydroxyprogesterone caproate
medroxyprogesterone acetate
megestrol acetate
Estrogens
diethylstilbestrol
ethynyl estradiol/equivalents
Antiestrogen
tamoxifen
Androgens
testosterone propionate
fluoxymesterone/equivalents
Antiandrogens
flutamide
gonadotropin-releasing
hormone analogs
leuprolide
Nonsteroidal antiandrogens
flutamide
Photosensitizers
hematoporphyrin derivatives
Photofrin ®
benzoporphyrin derivatives
Npe6
tin etioporphyrin (SnET2)
pheoboride-a
bacteriochlorophyll-a
naphthalocyanines
phthalocyanines
zinc phthalocyanines

Metal cations are also useful as T-type calcium channel inhibitors including, but not limited to yttrium (Y³⁺), gadolinium (Gd³⁺), erbium (Er³⁺), ytterbium (Yb³⁺), lanthanum (La³⁺), and nickel (Ni²⁺).

Mibefradil compositions contemplated by the invention may also include cytokines and growth factors that are effective in inhibiting tumor metastasis. Such cytokines, lymphokines, growth factors, or other hematopoietic factors include M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, erythropoietin and other known angiopoietins, for example Ang-1, Ang-2, Ang-4, Ang-Y, and/or the human angiopoietin-like polypeptide, and/or vascular endothelial growth factor (VEGF). Preferred growth factors for use in pharmaceutical compositions of the invention include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil, chemotactic factor 2 α, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

Compositions of the invention are also readily adaptable for use in assay systems, for example, assaying cancer cell growth and properties thereof using mibefradil compositions described herein, as well as identifying compounds that affect cancer cell growth and metastasis.

Formulation of Pharmaceutical Compositions

The methods of the invention are preferably carried out using a composition as described herein with one or more pharmaceutically acceptable carriers. The phrase “pharmaceutically or pharmacologically acceptable” refer to molecular entities and compositions that do not produce allergic, or other adverse reactions when administered using routes well-known in the art, as described below. “Pharmaceutically acceptable carriers” also include any and all clinically useful solvents, dispersion media, coatings, antibacterial and antifungal agents, isotonic and absorption delaying agents and the like. The use of such media and agents for pharmaceutically active substances is well known in the art.

Pharmaceutical carriers include pharmaceutically acceptable salts, particularly where a basic or acidic group is present in a compound. For example, when an acidic substituent, such as —COOH, is present, the ammonium, sodium, potassium, calcium and the like salts, are contemplated for administration. Additionally, where an acid group is present, pharmaceutically acceptable esters of the compound (e.g., methyl, tert-butyl, pivaloyloxymethyl, succinyl, and the like) are contemplated as preferred forms of the compounds, such esters being known in the art for modifying solubility and/or hydrolysis characteristics for use as sustained release or prodrug formulations.

When a basic group (such as amino or a basic heteroaryl radical, such as pyridyl) is present, then an acidic salt, such as hydrochloride, hydrobromide, acetate, maleate, pamoate, phosphate, methanesulfonate, p-toluenesulfonate, and the like, is contemplated as a form for administration.

In addition, compounds may form solvates with water or common organic solvents. Such solvates are contemplated as well.

The mibefradil compositions may be administered orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and or surgical implantation at a particular site is contemplated as well. Generally, compositions are essentially free of pyrogens, as well as other impurities that could be harmful to the recipient.

The pharmaceutical compositions containing mibefradil as described above are in a form suitable for oral use, for example, as tablets, troches, lozenges, aqueous or oily suspensions, dispersible powders or granules, emulsions, hard or soft capsules, or syrups or elixirs. Compositions intended for oral use are prepared according to any known method, and such compositions contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients can include, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for controlled release.

Formulations for oral use may be presented as hard gelatin capsules wherein the active ingredient is mixed with an inert solid diluent, for example, calcium carbonate, calcium phosphate or kaolin, or as soft gelating capsules wherein the active ingredient is mixed with water or an oil medium, for example peanut oil, liquid paraffin, or olive oil.

Aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

The pharmaceutical compositions of the invention may also be in the form of oil-in-water emulsions. The oily phase may be a vegetable oil, for example olive oil, arachis oil, sesame oil or coconut oil, or a mineral oil, for example liquid paraffin, or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous, oleaginous suspension, dispersions or sterile powders for the extemporaneous preparation of sterile injectable solutions or dispersions. The suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. The carrier can be a solvent or dispersion medium containing, for example, water, ethanol, polyol (for example, glycerol, propylene glycol, and liquid polyethylene glycol, and the like), suitable mixtures thereof, vegetable oils, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

In all cases the form must be sterile and must be fluid to the extent that easy syringability exists. The proper fluidity can be maintained, for example, by the use of a coating, such as lecithin, by the maintenance of the required particle size in the case of dispersion and by the use of surfactants. It must be stable under the conditions of manufacture and storage and must be preserved against the contaminating action of microorganisms, such as bacteria and fungi. The prevention of the action of microorganisms can be brought about by various antibacterial an antifungal agents, for example, parabens, chlorobutanol, phenol, sorbic acid, thimerosal, and the like. In many cases, it will be desirable to include isotonic agents, for example, sugars or sodium chloride. Prolonged absorption of the injectable compositions can be brought about by the use in the compositions of agents delaying absorption, for example, aluminum monostearate and gelatin.

The compositions may also be in the form of suppositories for rectal administration of the mibefradil composition. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols, for example.

Compositions useful for administration may be formulated with uptake or absorption enhancers to increase their efficacy. Such enhancer include for example, salicylate, glycocholate/linoleate, glycholate, aprotinin, bacitracin, SDS, caprate and the like. See, e.g., Fix (J. Pharm. Sci., 85:1282-1285, 1996) and Oliyai and Stella (Ann. Rev. Pharmacol. Toxicol., 32:521-544, 1993).

Mibefradil compositions contemplated for use inhibit cancer growth, including proliferation, invasiveness, and metastasis, thereby rendering them particularly desirable for the treatment of cancer. In particular, the compositions exhibit cancer-inhibitory properties at concentrations that are substantially free of side effects, and are therefore useful for extended treatment protocols. For example, co-administration of a mibefradil composition with another, more toxic, chemotherapeutic agent can achieve beneficial inhibition of a cancer, while effectively reducing the toxic side effects in the patient.

In addition, the properties of hydrophilicity and hydrophobicity of the compositions contemplated for use in the invention are well balanced, thereby enhancing their utility for both in vitro and especially in vivo uses, while other compositions lacking such balance are of substantially less utility. Specifically, compositions contemplated for use in the invention have an appropriate degree of solubility in aqueous media which permits absorption and bioavailability in the body, while also having a degree of solubility in lipids which permits the compounds to traverse the cell membrane to a putative site of action. Thus, mibefradil compositions contemplated are maximally effective when they can be delivered to the site of the tumor and they enter the tumor cells.

Administration and Dosing

In one aspect, methods of the invention include a step of administration of a pharmaceutical composition.

Methods of the invention are performed using any medically-accepted means for introducing a therapeutic directly or indirectly into a mammalian subject, including but not limited to injections, oral ingestion, intranasal, topical, transdermal, parenteral, inhalation spray, vaginal, or rectal administration. The term parenteral as used herein includes subcutaneous, intravenous, intramuscular, and intracisternal injections, as well as catheter or infusion techniques. Administration by, intradermal, intramammary, intraperitoneal, intrathecal, retrobulbar, intrapulmonary injection and or surgical implantation at a particular site is contemplated as well.

In one embodiment, administration is performed at the site of a cancer or affected tissue needing treatment by direct injection into the site or via a sustained delivery or sustained release mechanism, which can deliver the formulation internally. For example, biodegradable microspheres or capsules or other biodegradable polymer configurations capable of sustained delivery of a composition (e.g., a soluble polypeptide, antibody, or small molecule) can be included in the formulations of the invention implanted near the cancer.

Therapeutic compositions may also be delivered to the patient at multiple sites. The multiple administrations may be rendered simultaneously or may be administered over a period of time. In certain cases it is beneficial to provide a continuous flow of the therapeutic composition. Additional therapy may be administered on a period basis, for example, hourly, daily, weekly or monthly.

Particularly contemplated in the presenting invention is the administration of multiple agents, such as a T-type calcium channel inhibitor in conjunction with a second agent as described herein. It is contemplated that these agents may be given simultaneously, in the same formulation. It is further contemplated that the agents are administered in a separate formulation and administered concurrently, with concurrently referring to agents given within 30 minutes of each other.

In another aspect, the second agent is administered prior to administration of the T-type calcium channel inhibitor. Prior administration refers to administration of the second agent within the range of one week prior to treatment with the T-type calcium channel inhibitor, up to 30 minutes before administration of the T-type calcium channel inhibitor. It is further contemplated that the second agent is administered subsequent to administration of the T-type calcium channel inhibitor. Subsequent administration is meant to describe administration from 30 minutes after T-type calcium channel inhibitor treatment up to one week after T-type calcium channel inhibitor administration.

It is further contemplated that when mibefradil is administered in combination with a second agent, wherein the second agent is a second calcium channel inhibitor, a cytokine or growth factor, or a chemotherapeutic agent, the administration also includes use of a radiotherapeutic agent or radiation therapy. The radiation therapy administered in combination with a mibefradil composition is administered as determined by the treating physician, and at doses typically given to patients being treated for cancer.

The amounts of mibefradil in a given dosage of mibefradil composition will vary according to the size of the individual to whom the therapy is being administered as well as the characteristics of the disorder being treated. In exemplary treatments, it may be necessary to administer about 50 mg/day, 75 mg/day, 100 mg/day, 150 mg/day, 200 mg/day, 250 mg/day, 500 mg/day or 1000 mg/day. These concentrations may be administered as a single dosage form or as multiple doses. Standard dose-response studies, first in animal models and then in clinical testing, reveal optimal dosages for particular disease states and patient populations.

It will also be apparent that dosing should be modified if traditional therapeutics are administered in combination with therapeutics of the invention.

Kits

As an additional aspect, the invention includes kits which comprise one or more compounds or compositions packaged in a manner which facilitates their use to practice methods of the invention. In one embodiment, such a kit includes a compound or composition described herein (e.g., a composition comprising mibefradil alone or in combination with a second agent), packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the mibefradil composition.

Additional aspects and details of the invention will be apparent from the following examples, which are intended to be illustrative rather than limiting. Example 1 discloses that HT1080 cells express T-Type Ca²⁺ channels and TRP1 cation channels. Example 2 discloses that HT1080 cells exhibit Ca²⁺ oscillations that are blocked by mibefradil and gadolinium (Gd³⁺). Example 3 dicloses that the HT1080 Ca²⁺ spike is a perimembrane Ca²⁺ wave affected by mibefradil and Gd³⁺. Example 4 discloses inhibition of HT1080 cell migration by mibefradil and Gd³⁺. Example 5 discloses that mibefradil and Gd³⁺ inhibit HT1080 cell invasiveness. Example 6 describes methods of administration of mibefradil compositions to inhibit tumor metastasis in vivo. Example 7 describes methods for administering mibefradil compositions to inhibit tumor metastasis in cancer patients.

### EXAMPLE 1

### HT1080 Cells Express T-Type Ca²⁺ Channels and TRP1 Citation Channels

Previous studies show that treatment of cancer with either HVA calcium channel blockers or NVG calcium channel blockers does not completely inhibit tumor cell metastasis and invasion into distal tissues. This suggests that additional calcium channels are involved in tumor cell metastasis.

In order to determine if LVA (T-type) and NVG channels participate in the signaling mechanism(s) necessary for the migration of HT1080 fibrosarcoma cells, it was first necessary to confirm that structural elements of these channels are expressed by HT1080. The presence of T-type or NVG calcium channels on HT1080 cells was tested using immunofluorescence microscopy.

HT1080 cells were incubated in DMEM medium supplied with 10% FCS and 1% antibiotics. Cells were passaged by detachment from culture flasks with a trypsin/EDTA solution (Sigma, St. Louis, Mo.) in PBS for 5 min. at 37° C. For high speed imaging and emission microfluorometry, cells were pre-labeled with indo-1-AM (Molecular Probes, Inc., Eugene, Oreg.) at 5 μg/ml for 20 minutes at 37° C. (Kindzelskii et al., J. Immunol., 170:64-72, 2003). Anti-T-type channel antibody was prepared as described by using a peptide corresponding to amino acids 1 to 22 of the NH₂ terminal region of human α1G subunit of the low voltage T-type Ca²⁺ channel with addition of a C-terminal cysteine: NH₂-MDEEEDGAGAEESGQPRSFMRL(C)—COOH (SEQ ID NO: 1) (Andreasen et al., Am. J. Physiol. Renal Physiol. 279:F997-1005, 2000; Weiergraber et al., J. Histochem. Cytochem. 48:807-819, 2000). The purified peptide was conjugated to KLH then used to immunize rabbits (Bio-Synthesis Inc., Lewisville, Tex.). The labeled IgG fraction was found to specifically label cells.

Adherent HT1080 cells were fixed with 3.7% glutaraldehyde for 20 minutes at room temperature. Samples were washed and labeled with rabbit anti-T-type Ca²⁺ channel polyclonal antibody (2 μg/ml) or rabbit anti-TRPI cation channel polyclonal antibody (2 hg/ml) followed by labeling with a TRITC-conjugated goat ant-rabbit antibody (1 μg/ml). To minimize non-specific binding, 1% BSA was included in these solutions.

Fluorescence microscopy was performed with an Axiovert 135 fluorescence microscope with a high numerical aperture condenser, quartz objective, and an AttoArc mercury lamp (Zeiss). Images from multiple focal planes were collected using a z-scan apparatus (Veytek, Inc., Fairfield, Iowa) and a cooled Retiga 1300 camera (Q-Imaging, Burnaby, B.C., Canada) or an intensified charge-coupled device camera (Hamamatsu, Hamamatsu City, Japan). Images were managed, processed and deconvoluted using Supermicro Ultra 320 workstation with the software packages: Image-Pro, Microtome, Image-Scan, and Vox-Blast (Veytek, Inc.). A narrow bandpass filter set with excitation at 485DF20, emission at 530DF30 nm and a long-pass dichroic mirror at 510 nm were used.

Stacks of fluorescence micrographs obtained from individual cells followed by deconvolution analyses of the z-scans demonstrated that the T-type calcium channels were located primarily about the circumference of the cell and distributed randomly at the cell periphery. TRP1 staining was also primarily found at the cell periphery, exhibiting a punctate distribution. Labeling was often absent from pseudopods and may include a granule fraction near the plasma membrane. In addition, the z-scan/deconvolution image suggested that minor labeling of the ER was also observed with the anti-TRP1 reagent. This observation was consistent with the fact that the fixation protocol resulted in permeabilization of the cell.

Although prior studies have shown that tumor cells express T-type channel message, the present results identified the expression of TRP1 on tumor cells and T-type calcium channels on HT1080 cells. Thus, these studies confirmed the presence of antigens corresponding to LVA and NVG Ca²⁺ channels on these cells.

### EXAMPLE 2

### HT1080 Cells Exhibit Ca²⁺ Oscillations that are Blocked by Mibefradil and Gadolinium (GD³⁺)

Previous investigation of T-type calcium channels on 3T3 fibrobast cells demonstrated that these fibroblasts express both T-type and L-type calcium channels (Strobeck et al., J. Biol. Chem. 274:15694-700, 1999). Strobeck et al. (supra) demonstrated that expression of T-type calcium channels was suppressed in 3T3 fibroblast cells transformed by oncogenes. Additionally, administration of mibefradil to p53/ras-mutant transfected 3T3 cells induced morphological transformation of these rounded cells into spindle-shaped cells (Strobeck et al., supra), which are typically precursors to fibrosarcomas. Strobeck also teaches that T-type calcium channels exert greater control on fibroblast cell activity than L-type calcium channels.

The presence of T-type calcium channels on HT1080 cells, as disclosed above, indicates that these low-voltage-activated calcium channels may exert significant control on cell activity, similar to other fibroblast cell lines (Strobeck et al., supra). The suppression of T-type calcium channels on transformed 3T3 cells indicates these calcium channels may be involved in tumor cell development. Interestingly, HT1080 cells contain an activated N-ras oncogene, which may effect the regulation of T-type channel expression and activity, and promote morphogenesis into spindle-shaped cells in the presence of T-type calcium channel inhibitors. Thus, it was necessary to determine if blockade of T-type calcium channels on HT1080 cells caused morphological transformation.

In order to assess the activity of T-type channels in HT1080 cells in the presence of T-type calcium channel inhibitor, HT1080 cells were cultured with mibefradil and calcium levels were measured. To determine the extent and type of Ca²⁺ signal oscillations in HT1080 cells, cells were labeled with indo-1-AM as described above, 5 μg/ml for 20 minutes at 37° C. The HT1080 cells were suspended in HBSS then incubated at 37° C. for 20 minutes to allow the labeled cells to adhere to glass coverslips. Indo-1-AM is a fluorescent indicator dye that by itself does not bind calcium, but is hydrolyzed to indo-1 once inside cells. The indo-1 molecule binds calcium, and is thus an indicator of intracellular calcium levels.

Microfluorometry of untreated polarized cells showed a sustained series of Ca²⁺ spikes at 20 second intervals. To observe the longitudinal properties of these Ca²⁺ spikes, a long-time base was used. However, to observe the temporal details of each Ca²⁺ spike, data were also acquired using a short time base. A calcium trace of an untreated HT1080 cell shows a typical Ca²⁺ spike profile consisting of a low-intensity shoulder on the leading edge, a brief peak, and a decay in indo-1 intensity. No changes in Ca²⁺ spike frequency or amplitude were observed when cells were treated with 15 μM SKF96365, 1 μM diltiazem, 4 μM verapamil, and 1 μM nicardipine (Sigma Chemical Co., St. Louis, Mo.). These concentrations were chosen as they correspond to the levels associated with specific Ca²⁺ channel blockage; for example, nicardipine is an HVA (L-type) Ca²⁺ channel blocker effective in vitro at this concentration (Hagiwara et al., Neurosci. Res. 46:493-497, 2003). The NVG channel blocker and anti-metastatic agent CAI (Drug Synthesis & Chemistry Branch, Developmental Therapeutics Program, Division of Cancer Treatment and Diagnosis, NCI, Bethesda, Md.), employed at 10 μM for these in vitro studies (Rust et al., Anal. Biochem. 280:11-19, 2000), reduced Ca²⁺ spikes to Ca²⁺ “bumps,” which was then followed by elimination of the Ca²⁺ signal.

In a similar experiment, the T-type channel blocker mibefradil (1.5 μM) was added to the HT1080 cells at approximately one minute. Within approximately 3 to 4 minutes the fluorescent Ca²⁺ spikes were replaced by very low intensity bumps, which then disappeared. Similar results were obtained using Gd 3+, a specific TRP (Trebak et al., J. Biol. Chem. 277:21617-21623, 2002) and stretch-activated cation channel blocker (Yang et al., Science 243:1068-1071, 1989). Again, the Ca²⁺ spikes diminished then disappeared. When the Ca²⁺ bumps were evaluated using the short time base settings, these experiments demonstrated that Ca²⁺ bumps closely resemble the intensity shoulder previously noted for untreated HT1080 cells.

HT1080 cells treated with mibefradil expressed the same level of T-type calcium channel as untreated cells, indicating that mibefradil did not cause the channels to be downregulated, but inhibited their activity. Additionally, mibefradil treated HT1080 cells maintain their rounded shape, indicating that the cells are not morphologically transformed by administration of mibefradil, as suggested by Strobeck et al. (supra).

These results indicated that, unlike the results in 3T3 cells, mibefradil effectively inhibits calcium channel activity without inducing further transformation of HT1080 tumor cells.

### EXAMPLE 3

### The HT1080 Ca²⁺ Spike is a Perimembrane Ca²⁺ Wave Affected by Mibefradil and Gd³⁺

Recent studies have shown that Ca²⁺ spikes of polarized neutrophils and colorectal tumor cell transfectants are associated with certain intracellular Ca²⁺ waves (Kindzelskii et al., J. Immunol. 170:64-72, 2003; Wang et al., J Immunol. 170:795-804, 2003). To visualize these waves, images must be collected using very short shutter speeds to avoid image blurring due to wave motion and indo-1 diffusion while the shutter is open. A high speed imaging method (Kindzelskii et al., supra; Wang et al., supra, Worth et al., Proc. Natl. Aced. Sci. USA, 100:4533-4538, 2003; Kindzelskii et al., Proc. Natl. Acad. Sci. USA 99:9207-9212, 2002; Petty et al., Phys. Rev. Lett. 84:2754-2757, 2000) was used to detect these waves in HT1080 cells.

High speed microscopy was performed using an Axiovert 135 fluorescene microscope with a quartz condenser, quartz objective, and an AttoArc mercury lamp (Zeiss). To detect fluorescence changes in the short wavelength emission region of indo-1, a 355HT15 exciter, a 390LP dichroic reflector, and a 405DF43 emission filter were used. To increase light collection efficiency, the microscope's bottom port was used. This port was fiber-optically coupled to the input of an Acton-150 (Acton Instruments, Acton, Mass.) imaging spectrophotometer. The exit side was connected to a liquid N₂-cooled intensifier attached to a Peltier-cooled I-MAX-512 camera (˜−20° C.) (Princeton Instruments, Trenton, N.J. (Wang et al., supra, Worth et al., supra; Kindzelskii et al. (2002), supra). A Gen-II tube was used to provide maximal efficiency in the violet-blue region of the spectrum (Kindzelskii et al., (2003) supra), where indo-1 emits. The camera was controlled by a high speed Princeton ST-133 interface and a Stanford Research Systems (Sunnyvale, Calif.) DG-535 delay gate generator (Petty et al., Phys. Rev. Lett. 84:2754-2757, 2000). A custom-built computer with dual 3.06 GHz Xenon processors with 1 MB onboard cache each, 3.0-Gb RAM, 3.2 Tb of hard drive space with 64 MB cache, and a RAID-5 hard drive management system was used. For experiments, 2 Gb of RAM was allocated as a RAM disk. Winspec-32 (version 2.5.14.1; Princeton Instruments) software was used with a PCI communication accelerator. To improve computer acquisition times, the size of the pixel detection array of the CCD chip was adjusted using a Virtual-Chip plug-in. Winspec CPU calls were given system priority to enhance the instrument's duty cycle. Data were acquired without reporting to the monitor to further improve system speed. Data capture used a software-allocated disk.

High speed microscopy showed that the Ca²⁺ spike in untreated HT1080 cells was a counterclockwise wave in the microscope's frame of reference Ca²⁺. A region of high Ca concentration, seen in the microscope as a bright spot within the cell, was brought about by the local opening of Ca²⁺ channels. As indo-1 has a relatively high affinity for Ca²⁺ and a finite amount of time was required for the signaling apparatus to pump-down free cytosolic Ca²⁺, a low intensity “tail” was observed next to the region of bright Ca²⁺ indo-1 fluorescence (i.e., the Ca²⁺ wave has a low intensity tail on the side opposite to the direction of wave motion). This wave was found to travel with a velocity of 200 μm/sec. However, after addition of mibefradil, a brief Ca²⁺ spark was observed at the lamellipodium, although no propagating Ca²⁺ wave was found. Similarly, a Ca²⁺ wave was observed in cells before, but not after addition of Gd³⁺.

These results showed that the regular Ca²⁺ spikes observed in migrating tumor cells were counterclockwise waves, when viewed from the bottom of the cell on a coverslip. Furthermore, the shoulder of the temporal Ca²⁺ spike likely corresponded to the temporal bump and the spatiotemporal spark noted above. These waves were blocked by mibefradil and Gd³⁺, which suggests that their binding sites, including LVG and NVG channels, participate in wave propagation around the cell and ignition of the wave at the level of the plasma membrane. Inhibition of HT1080 calcium wave propagation by mibefradil and Gd³⁺ suggests that blockade of the calcium channel activity blocks an initial event necessary for cell movement, and that treatment with mibefradil may inhibit the cell's ability to migrate.

### EXAMPLE 4

### Mibefradil and Gd³⁺ Inhibit HT1080 Migration

The central role of Ca²⁺ and Ca²⁺ oscillations in cell motility and the ability of mibefradil and Gd³⁺ to influence these oscillations and waves suggest that these reagents may affect tumor cell motility. To assess the migratory ability of HT1080 tumor cells in the presence of calcium channel inhibitors, the phagokinetic track assay was used to test the ability of mibefradil and Gd³⁺ to inhibit HT1080 cell motility (Albrecht-Buehler, G. Cell 11:395-404, 1977; Scott et al., Anal. Biochem. 287:343-344, 2000.)

HT1080 cells were incubated on gold particle-coated glass coverslips for 22 hours. Coverslips were dipped into a 0.17% gelatin solution then coated with a colloidal gold layer. A suspension of 5000 cells/ml was treated with or without Ca²⁺ channel blockers then seeded onto the gold-coated coverslips. After 22 hours of incubation, the samples were fixed with 3.7% glutaraldelryde. The tracks produced by the cells were viewed by brightfield microscopy with a 10× objective. Thirty randomly selected tracks were measured for each slide.

As cells migrated on these surfaces, the gold particles were cleared from regions of the surface and frequently picked up by the cells, making the cells appear darker and the surrounding area appear lighter when viewed by microscopy. Micrographs of HT1080 cells demonstrated that in the absence of added reagents, large areas of the surface were cleared by the cells, as the cells become darker. As detected by brightfield microscopy, culture with 10 μM CAI, 1.5 μM mibefradil, or 1.5 μM Gd³⁺ significantly decreased the ability of HT1080 cells to clear the surrounding surface. However, at concentrations of SKF96365, diltiazem, verapamil, and nicardipine expected to block the relevant Ca²⁺ channels, no inhibitory effect was observed.

The size of HT1080 cells varies considerably. To evaluate cell motility, a swept area ratio was used, which calculates total swept area divided by cell area. Measurement of this ratio reiterated that mibefradil and Gd³⁺ significantly inhibited cell motility in a dose-dependent manner. Six μM Gd³⁺ almost completely abolished HT1080 movement, and 5 μM mibefradil decreased the cell-swept area approximately 90% compared with control treated cells. Thus, qualitative data indicated that blockade of LVG and NVG channels, but not L-type Ca²⁺ channels, diminished HT1080 cell motility.

These results demonstrated that mibefradil inhibition of T-type calcium channels is an effective method to significantly reduce tumor cell motility in vitro, and provides a potential therapy for inhibition of tumor cells in vivo.

### EXAMPLE 5

### Mibefradil and Gd³⁺ Inhibit HT1080 Cell Invasiveness

Cell motility is required for invasion of tumor cells into areas outside the original tumor location. To determine the effects of calcium channel inhibitors on HT1080 tumor cell invasiveness, the ability of mibefradil and Gd³⁺ to inhibit HT1080 cell invasion was tested.

Biocoat Matrigel invasion chambers (BD Biosciences, Bedford, Mass.) were used for HT1080 cell invasion experiments. The chambers contained an 8-μm pore size membrane with a thin Matrigel basement membrane matrix as previously described (Wang et al., J Immunol. 170:795-804. 2003). One-half ml of cells (5×10⁴ cells/ml) in serum-free DMEM medium with or without Ca²⁺ channel blockers were added to the cell culture insert of a Biocoat Matrigel invasion chamber. Fibronectin (25 μg/ml) was added in the outer chamber as a chemoattractant. The cells were then incubated at 37° C. in humidified 5% CO₂ for 22 hours. To quantitate tumor cell invasion, noninvading cells were removed from the upper surface of the membrane by scrubbing gently with a cotton-tipped swab. The cells on the lower surface of the membrane were fixed with Diff-Quik stain set (Dade Behring, Newark, Del.) and counted to determine the number of cells that passed through the Matrigel and membrane layers.

Cell suspensions were loaded to the upper chamber with or without the addition of mibefradil, Gd³⁺, nicardipine or other reagents. After a 22 hour incubation period, transmigrated cells on the underside of the inserts were fixed, stained and counted. Both mibefradil and Gd³⁺ inhibited HT1080 cell transmigration in a dose-dependent fashion. The number of migrating cells decreased from approximately 80 in control cells to approximately 25 in mibefradil treated cells and approximately 22 in Gd3+ treated cells. Nicardipine, SKF96365, verapamil, and diltiazem had no effect on cell invasiveness at pharmacologically appropriate doses. Moreover, when the lowest doses of mibefradil and Gd³⁺ that exhibit maximal inhibition of invasiveness (5 and 20 μM, respectively) were employed simultaneously, the drug combination further augmented HT1080 inhibition by up to 95%, allowing only 6 transmigrating cells. The complementary action of these two reagents was consistent with their ability to act on two different membrane sites contributing to the same physical wave. Similarly, culture of HT1080 cells with mibefradil plus CAI showed a significant reduction in invasiveness in comparison to mibefradil alone, decreasing the number of migrating cells to from approximately 25 to approximately 12.

These results demonstrated that not only cell motility, but also cellular invasion of HT1080 cells, was regulated by T-type and NVG Ca²⁺ channels, and not by L-type Ca²⁺ channels.

### EXAMPLE 6

### Administration of Mibefradil Compositions to Inhibit Tumor Metastasis In Vivo

Calcium channel blockers that inhibit cell signaling through high voltage activated calcium channels or through non-voltage gated calcium channels have been used previously in attempts to reduce or inhibit tumor cell metastasis and invasion into distal tissue sites. To determine the effects of low-voltage gated calcium channels and inhibitors thereof, e.g. mibefradil, on tumor metastasis, animal models of tumor metastasis are used.

Experimental models known in the art to induce development of metastatic tumors are useful for assessing the effects of mibefradil on tumor metastasis. Animal models of cancer metastasis useful in assessing mebefradil acitivty include animal models for gastric cancer (Illert et al., Clin. Exp. Metastasis 20:549-54, 2003), colon cancer (Sturm et al., Clin. Exp. Metastasis 20:395-405, 2003), pancreatic cancer (Katz et al., J. Surg. Res. 113:151-60, 2003), prostate cancer (Bastide et al., Prostate Cancer Prostatic Dis. 5:311-15, 2002), lymphogenic metastasis (Dunne et al., Anticancer Res. 22:3273-9, 2002), human lymphoma metastasis (Aoudjit, et al., J. Immunol., 161:2333-2338, 1998), breast cancer (Li et al., Clin. Exp. Metastasis 19:347-50, 2002; Pulaski et al., Cancer Res. 60:2710-15, 2000), colorectal cancer (Kuruppu et al., J. Gastroenterol. Hepatol. 13:521-7, 1998), hepatocellular carcinoma, (Lindsay et al., Hepatology 26:1209-15, 1997), neuroblastoma (Engler et al., Cancer Res. 61:2968-73, 2001), and fibrosarcoma metastasis (Shioda et al., J. Surg. Oncol. 64:122-6, 1997; Culp et al., Prog. Histochem. Cytochem. 33:XI-XV, 329-48, 1998).

As an example, mice are inoculated with 7×10³ breast tumor cells or wild type cells as described in Pulaski et al. (supra). Primary breast tumors allowed to grow in an animal for 2-3 weeks typically demonstrate metastasis in the lung, lymph node and liver. The percentage of cells invading these sites increases over time. The tumors are allowed to grow for 2-3 weeks and animals are then treated with appropriate doses, pre-determined by one of skill in the art, of a mibefradil composition set out herein, such as mibefradil alone, mibefradil plus CAI, mibefradil plus gadolinium, gadolinium alone, or a control agent. Mibefradil may be administered in 100 μg/kg bolus (J Pharmacol Exp Ther. 285: 746-52, 1998). CAI may be administered in a range of 50-150 mg/m² (Berlin et al., Clin Cancer Res. 8:86-94, 2002). Gadolinium may be administered to animals in a range of 0.5 mg/kg to 50 mg/kg (Ding et al., World J Gastroenterol. 9:1072-6, 2003; Harstad et al., Toxicol Appl Pharmacol. 180:178-85, 2002). When the agents are administered in combination with one another, the dosage administered to an animal may be adjusted accordingly.

Mibefradil alone is administered to mice given tumorigenic cells either concurrent with inoculation of tumor cells, or 7, 14, 21 or 28 days after induction. It is contemplated that mibefradil is given either in one dose, in two doses, daily for an extended period of time, e.g., 7 or 14 days, or supplied continuously in the drinking water of treated animals, with the dose administered in terms of consumption of water/day.

When mibefradil is given in conjunction with a second calcium channel blocker, e.g., CAI or gadolinium, the mibefradil may be administered concurrent with the second calcium channel blocker, prior to the second calcium channel blocker or subsequent to the second calcium channel blocker. The agents may be administered in the dose regimen described above, or in another dose regimen predetermined to be effective.

Tumor growth in treated and untreated animals is assessed by measuring tumor diameter before and after treatment with mibefradil. Tumor diameter is measured weekly after inoculation with tumor cells. A decrease in tumor diameter in the mibefradil treated mice demonstrates that blockade of T-type calcium channels inhibits tumor growth and that calcium signaling through low voltage activated receptors is important for tumor growth.

To measure metastasis, the primary tumor is removed in one set of animals or permitted to continue growing in a second group of animals. Mice administered tumor cells demonstrate metastasis when the inoculating cells are isolated in a tissue or site away from the initial induction site. Animals are sacrificed at varying timepoints over the course of treatment (e.g. day 7, day 14, day 21, day 24, day 28, and day 35) and histologic examination of tissue slices from lung, lymph nodes, liver, kidney and other organs analyzed for the presence of metastasized cells using cell surface markers specific for the tumor cell.

A decrease in metastasis in the mibefradil treated animals shows that T-type calcium channels are involved in the cell signaling mechanism that regulates tumor cell metastasis and invasion into secondary tissues. Treatment of tumorigenic animals with the combination of mibefradil and either CAI or gadolinium will likely provide a synergistic anti-metastatic effect and decrease the extent of tumor metastasis over that of mibefradil treatment alone.

### EXAMPLE 7

### Administration of Mibefradil Compositions to Inhibit Tumor Metastasis in Cancer Patients

Administration of mibefradil in animal models of tumor metastasis provides the basis for administering cancer patients mibefradil alone or in combination with other calcium channel blockers, cytokines or growth factors, or chemotherapeutic or radiotherapeutic agents. Mibefradil is administered using regimens similar to those described for administration of the NVG calcium channel inhibitor, CAI (Bauer et al., Clin. Cancer Res. 5:2324-2329, 1999; Kohn et al., Clin. Cancer Res. 7:1600-1609, 2001).

Mibefradil is administered to patients at a dose of 50 mg/day. It is recognized by one of skill in the art that the amount of dose will vary from patient to patient, and may be anywhere from 20 mg/kg/day to 500 mg/kg/day. Mibefradil is administered in doses appropriate for the patient's size, sex, and weight, as would be known or readily determined in the art. Subsequent doses of the inhibitor may be increased or decreased to address the particular patient's response to therapy.

Mibefradil is given in any formulation recognized in the art to allow mibefradil to diffuse into the bloodstream or tissue sites, e.g. capsule form, micronized form, or liquid form. Mibefradil is administered at a frequency and dose determined by the treating physician. For example, mibefradil may be administered once daily for 7 days, twice daily for 7 days, every other day for 14 days, continuously for 14 days, or any other regiment the physician prescribes. Mibefradil may be administered continuously, e.g., through intravenous delivery or by slow release methods, for an extended period of time. The administration may last 4-24 hours, or longer and is amenable to optimization using routine experimentation. The calcium channel inhibitor may also be given for a duration not requiring extended treatment. Additionally, the calcium channel inhibitor may be administered daily, weekly, bi-weekly, or at other effective frequencies, as would be determinable by one of ordinary skill in the art.

Mibefradil is administered to patients in combination with other therapeutics, such as a second calcium channel inhibitor, for example, CAI and Gd³⁺, with other chemotherapeutic or radiotherapeutic agents, or with growth factors or cytokines. When given in combination with another agent, the amount of mibefradil given may be reduced accordingly. CAI may be administered, in conjunction with mibefradil, in an amount as described by Kohn et al. (supra) or Bauer et al. (supra). Gadolinium may be administered, in conjunction with mibefradil, in an dose range from 0.3 mg/kg to 8.4 mg./kg. For example, in one aspect, gadolinium is administered in a range of 5 mg/kg to 6.3 mg/kg (Carde et al., J Clin Oncol. 19:2074-83, 2001). Other agents are administered in an amount determined to be safe and effective at ameliorating human disease.

It is contemplated that second calcium channel inhibitors, cytokines or growth factors, and chemotherapeutic agents or radiotherapeutic agents are administered in the same formulation as mibefradil and given simultaneously. Alternatively, the agents may also be administered in a separate formulation and still be administered concurrently with mibefradil, concurrently referring to agents given within 30 minutes of each other. The second agent may also be administered prior to administration of mibefradil. Prior administration refers to administration of the agent within the range of one week prior to mibefradil treatment up to 30 minutes before administration of mibefradil. It is further contemplated that the second agent is administered subsequent to administration of mibefradil. Subsequent administration is meant to describe administration from 30 minutes after mibefradil treatment up to one week after mibefradil administration. Mibefradil compositions may also be administered in conjunction with a regimen of radiation therapy as prescribed by a treating physician.

In one approach, the effectiveness of mibefradil treatment is determined by computer tomographic (CT) scans of the tumor area with the degree of tumor regression assessed by measuring the decrease in tumor size. Biopsies or blood samples are also used to assess the presence or absence and metastasizing ability of particular cell types in response to treatment with mibefradil alone, or in combination with other chemotherapeutic agents. These response assessments are made periodically during the course of treatment to monitor the response of a patient to a given therapy.

A decrease in tumor size, reduction of tumor metastasis and improvement in patient prognosis after treatment with mibefradil alone or in combination with a second calcium channel inhibitor or other chemotherapeutic agent indicates that the method effectively treats patients exhibiting tumor metastasis or tumors capable of tumor metastasis. Calcium channel inhibitors or chemotherapeutic agents used in conjunction with mibefradil and identified by the methods disclosed herein as effective against tumor metastasis are expected to be therapeutically useful in the inhibition or reduction of tumor metastasis.

As noted above, several isoforms of T-type calcium channels exist, each possessing a different kinetic and pharmacologic profile. This presents a difficulty in developing a single T-type calcium channel inhibitor that effectively blocks activity of all T-type calcium channel isoforms. Moreover, a single isoform may be expressed differently on multiple cell types, adding a degree of complexity to developing universal inhibitors of these low-voltage-activated calcium channels. The present invention contemplates a method for determining a regimen for treating cancer in a subject that is specific for that individual, and more suited to treat a particular form of cancer metastasis.

Tumor cells isolated from an individual exhibiting tumor metastasis or a tumor type at risk for metastasis are cultured in vitro with or without a mibefradil composition in order to determine if the mibefradil composition tested is effective at inhibiting cell activity in the particular tumor type. Effectiveness of the mibefradil composition against the tumor is measured using methods described in above, such as cell toxicity assays, calcium flux assays, and migration assays. When a therapeutically effective mibefradil composition is identified for the subject undergoing testing, the mibefradil composition may then be administered to the patient in an amount effective to ameliorate or inhibit tumor metastasis.

Numerous modifications and variations in the invention as set forth in the above illustrative examples are expected to occur to those skilled in the art. Consequently only such limitations as appear in the appended claims should be placed on the invention.

We claim:

1. A method of inhibiting tumor cell metastasis in cancer comprising the step of administering a T-type calcium channel inhibitor in an amount effective to inhibit tumor cell metastasis.

2. The method of claim 1 wherein the T-type calcium channel inhibitor is selected from the group consisting of a diphenyubutylpiperidine, a hormone or hormonal regulator, a GABA agonist, an antiepileptic agent, an antipsychotic agent, and efonidipine.

3. The method of claim 2 wherein the T-type calcium channel inhibitor is selected from the group consisting of mibefradil, nickel (Ni²⁺), baclofen, flunarizine, nicardipine, felodipine, amiloride, ethosuximide, methylphenylsccinimide, phenyloin, zonisamide, and penfluridol.

4. The method of claim 3 wherein the T-type calcium channel inhibitor is mibefradil.

5. The method of claim 1 wherein the T-type calcium channel inhibitor is administered in combination with a cytokine or growth factor.

6. The method of claim 1 wherein the T-type calcium channel inhibitor is administered in conjunction with a chemotherapeutic or radiotherapeutic agent.

7. The method of claim 1 wherein the T-type calcium channel inhibitor is administered in combination with a NVG calcium channel inhibitor.

8. The method of claim 7 wherein the NVG channel inhibitor is selected from the group consisting of carboxyamidotriazole (CAI), gadolinium (Gd³⁺), SKF-96365, and lanthanum (La³⁺), and econazole.

9. The method of claim 7 wherein the T-type calcium channel inhibitor is mibefradil and the NVG calcium channel inhibitor is gadolinium (Gd³⁺).

10. The method of claim 7 wherein the T-type calcium channel inhibitor is mibefradil and the NVG calcium channel inhibitor is carboxyamidotriazole (CAI).

11. The method of claim 7 wherein the T-type calcium channel inhibitor and the NVG calcium channel inhibitor are administered in combination with a cytokine or growth factor.

12. The method of claim 7 wherein the T-type calcium channel inhibitor and the NVG calcium channel inhibitor are administered in combination with a chemotherapeutic or radiotherapeutic agent.

13. The method of any one of claims 1-12 wherein the cancer is fibrosarcoma.

14. A method for designing a treatment regimen for a patient with tumor cell metastasis comprising the steps of:
(a) isolating a cell from said patient, wherein said cell comprises a T-type calcium channel;
(b) contacting said cell with a mibefradil composition
(c) detecting T-type calcium channel activity in said cell to determine the amount of T-type calcium channel activity in the presence and in the absence of the mibefradil composition; and
(d) designing a treatment regimen for said patient which includes administration of mibefradil composition that specifically inhibits a T-type calcium channel activity in said patient.
